(19) United States
(12) Patent Application Publication
(10) Pub. No.: US 2020/0390537 A1
(43) Pub. Date: Dec. 17, 2020

(54) Endobronchial Blocker for Treatment of Persistent Air Leaks

(71) Applicant: Deepankar Sharma, Columbus, IN (US)

(72) Inventor: Deepankar Sharma, Columbus, IN (US)

(21) Appl. No.: 16/441,975

(22) Filed: Jun. 14, 2019

(51) Int. Cl. A61F 2/04; A61B 17/12

(52) U.S. Cl. CPC A61F 2/04; A61F 2002/043; A61B 17/12104; A61B 17/12031

(57) ABSTRACT

An endobronchial blocker for treatment of a persistent air leak, the endobronchial blocker comprising a perimeter wall, the perimeter wall comprising an exterior surface and an interior surface and enclosing a hollow internal cavity; an open end, the open end comprising a rim defining an opening to the hollow internal cavity; a closed end opposite the open end, the closed end and the perimeter wall being airtightly arranged so as to obstruct airflow into and out of the hollow internal cavity; and one or more friction enhancing elements on the exterior surface of the perimeter wall.

### BACKGROUND

Persistent air leaks are a cause of significant morbidity in patients who have undergone lung surgery and those with significant parenchymal lung disease or suffering from a pneumothorax. Although most air leaks resolve spontaneously with conservative management, some leaks are prolonged and contribute to significant morbidity. When a persistent air leak will not resolve, practitioners have long resorted to surgical management and chemical pleurodesis as the usual treatments for persistent air leaks. Although surgery is an effective means of treating persistent air leaks with relatively low mortality, resulting morbidity is much higher. There exists a need for less morbid treatment options as well as options for patients who are not operable.

Accordingly, there exists a need for an effective alternative for treating persistent air leaks, and in particular an apparatus and method of using the apparatus that can be customized to the patient's individual needs such that all patients can benefit.

### SUMMARY

The present disclosure includes disclosure of an endobronchial blocker for treatment of a persistent air leak and method of using the same. In at least one embodiment, such an endobronchial blocker comprises a perimeter wall with an exterior surface and an interior surface and enclosing a hollow internal cavity; an open end comprising a rim defining an opening to the hollow internal cavity; a closed end opposite the open end, airtightly arranged with the perimeter wall being so as to obstruct airflow into and out of the hollow internal cavity; and one or more friction enhancing elements on the exterior surface of the perimeter wall. In an aspect of such an embodiment, the perimeter wall comprises a flexible biocompatible material. In a further aspect of such an embodiment, the flexible biocompatible material comprises an implant-grade silicone. In an aspect of such an embodiment, at least one physical characteristic of the perimeter wall is selected as a result of a patient's pre-procedural testing. In a further aspect of such an embodiment, the physical characteristic is selected from the group consisting of width, length, shape, and wall thickness. In an aspect of such an embodiment, the perimeter wall is cylindrical. In an aspect of such an embodiment, the perimeter wall is hour glass shaped. In an aspect of such an embodiment, the perimeter wall further comprises at least one branch. In an aspect of such an embodiment, the friction enhancing elements comprise a plurality of studs distributed on the exterior surface.

In at least one embodiment, such a method of treating a persistent air leak by inserting an endobronchial blocker comprises the steps of selecting a site for placement of said endobronchial blocker to treat a patient's persistent air leak by identifying an airway or a bronchus leading to said persistent air leak, and measuring at least one physical characteristic of said airway, said bronchus, or said persistent air leak; providing an endobronchial blocker, the endobronchial blocker comprising: a perimeter wall with an exterior surface and an interior surface and enclosing a hollow internal cavity, an open end, comprising a rim defining an opening to the hollow internal cavity, a closed end opposite the open end, airtightly arranged with the perimeter wall being so as to obstruct airflow into and out of the hollow internal cavity; and one or more friction enhancing elements on the exterior surface of the perimeter wall; loading the endobronchial blocker into a bronchoscope; inserting the bronchoscope into the patient's upper airway; maneuvering the bronchoscope to location of the bronchus or airway selected for placement of the endobronchial blocker; releasing the endobronchial blocker from the bronchoscope in position to occlude the persistent air leak; monitoring clinical measurements to ensure the endobronchial blocker has reduced or eliminated said persistent air leak; and securing the endobronchial blocker in the patient's airway. In an aspect of such an embodiment, the perimeter wall comprises a flexible biocompatible material. In a further aspect of such an embodiment, the flexible biocompatible material comprises an implant-grade silicone. In an aspect of such an embodiment, at least one physical characteristic of the perimeter wall is selected as a result of a patient's pre-procedural testing. In a further aspect of such an embodiment, the physical characteristic is selected from the group consisting of width, length, shape, and wall thickness. In an aspect of such an embodiment, the perimeter wall is cylindrical. In an aspect of such an embodiment, the perimeter wall is hour glass shaped. In an aspect of such an embodiment, the perimeter wall further comprises at least one branch. In an aspect of such an embodiment, the friction enhancing elements comprise a plurality of studs distributed on the exterior surface. In an aspect of such an embodiment, the persistent air leak is caused by a fistula. In an aspect of such an embodiment, the bronchoscope comprises a rigid bronchoscope.

### BRIEF DESCRIPTION OF DRAWINGS

The embodiments and other features, advantages and disclosures contained herein, and the manner of attaining them, will become apparent and the present disclosure will be better understood by reference to the following description of various exemplary embodiments of the present disclosure taken in conjunction with the accompanying drawings, wherein:

FIG. 1 shows a perspective view of an endobronchial blocker according to at least one embodiment of the present disclosure;

FIG. 2 shows a perspective view of an endobronchial blocker according to at least one embodiment of the present disclosure;

FIG. 3 shows an end view of an endobronchial blocker according to at least one embodiment of the present disclosure;

FIG. 4 shows an end view of an endobronchial blocker according to at least one embodiment of the present disclosure;

FIG. 5 shows a cross-sectional view of an endobronchial blocker according to at least one embodiment of the present disclosure taken on line V-V of FIG. 3;

FIG. 6 shows a flowchart illustrating a method of using an endobronchial blocker according to at least one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

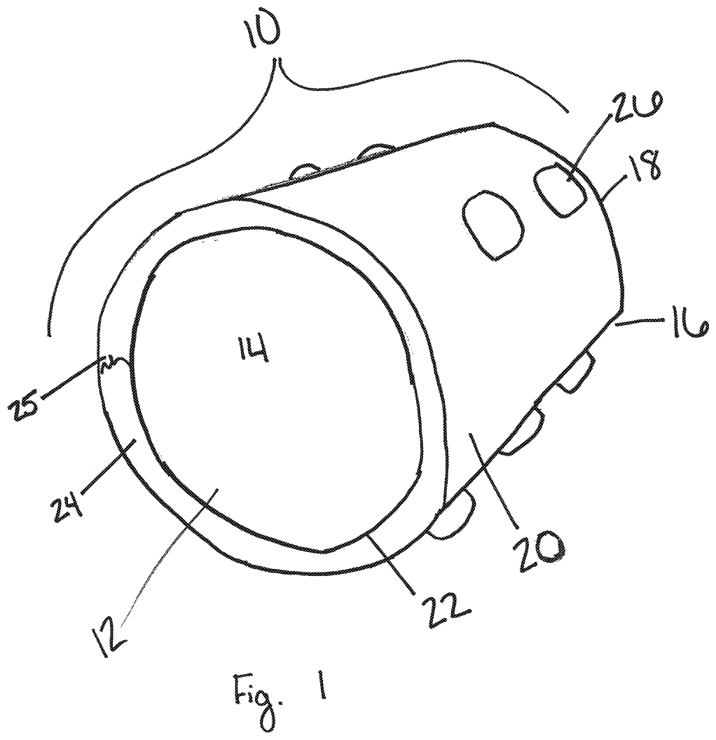

FIG. 1 shows an endobronchial blocker 10 according to at least one embodiment of the present disclosure. An endobronchial blocker 10 of the present disclosure can be used by medical personnel as a treatment for persistent air leaks, such as those caused by bronchopleural fistulas and alveolar pleural fistulas. Endobronchial blocker 10 according to the present disclosure can be made of any flexible biocompatible material suitable for use in a living organism, which in a particular, non-limiting embodiment is an implant-grade silicone.

As shown in FIG. 1, an endobronchial blocker 10 according to at least one embodiment of the present disclosure is a hollow tube 16 having an open end 12, closed end 18, and continuous hollow cylinder 14 between the two ends. Tube 16 has an exterior surface 20 and an interior surface 22 that define the perimeter wall 24 and diameter of the hollow cylinder 14. Perimeter wall 24 extends the length of hollow cylinder 14 and terminates in rim 25, which defines open end 12. Interior surface 22 is preferably smooth, while exterior surface 20 is preferably treated to provide higher friction between exterior surface 20 and the adjacent pulmonary tissue. Endobronchial blocker 10 according to at least one embodiment of the present disclosure comprises one or more friction enhancing elements 26 on the exterior surface 20 of tube 16. The friction enhancing elements 26 assists in keeping the endobronchial blocker 10 in place once inserted into a human subject. In a particular, non-limiting embodiment, the friction enhancing elements 26 comprise raised studs distributed on exterior surface 20.

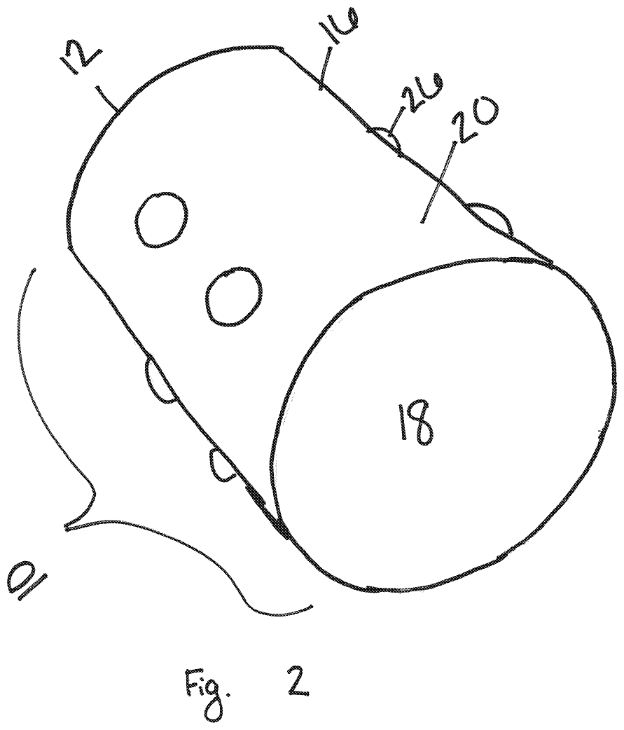

FIG. 2 shows a perspective view of an endobronchial blocker 10 according to at least one embodiment of the present disclosure. Shown in FIG. 2 is hollow tube 16 comprising friction enhancing elements 26 on the exterior surface 20 and closed distal end 18. Closed end 18 is made from made of any flexible biocompatible material suitable for use in a living organism, which in a non-limiting embodiment is the same material that hollow tube 16 is made from. In a particular non-limiting embodiment, this material comprises an implant-grade silicone. In at least one embodiment of the present disclosure, endobronchial blocker 10 can be manufactured by molding a predetermined amount of a biocompatible material into the desired shape, whereby the junction between hollow tube 16 and closed end 18 is seamless and airtight. In another embodiment of the present disclosure, endobronchial blocker 10 may be created by affixing an independent closed end 18 onto hollow tube 16 in an airtight manner. After considering the present disclosure, it will become apparent that endobronchial blocker 10 may be manufactured via any method that results in hollow tube 16 and closed end 18 forming an airtight connection so as to obstruct air flow for treatment of persistent air leaks.

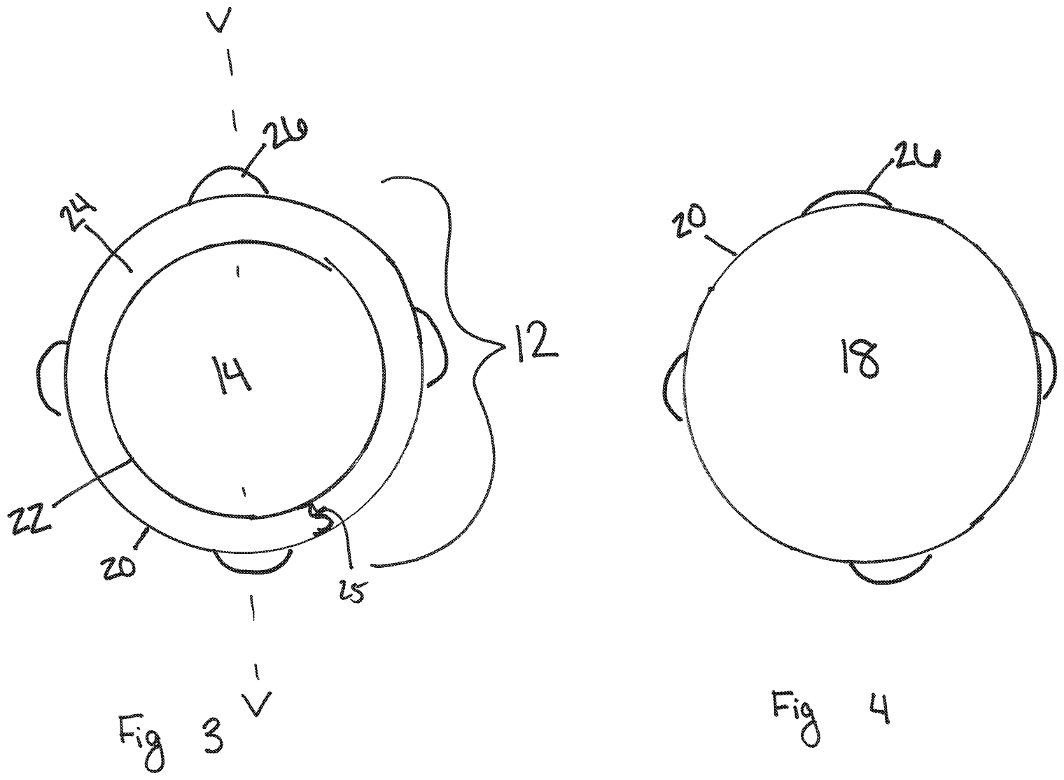

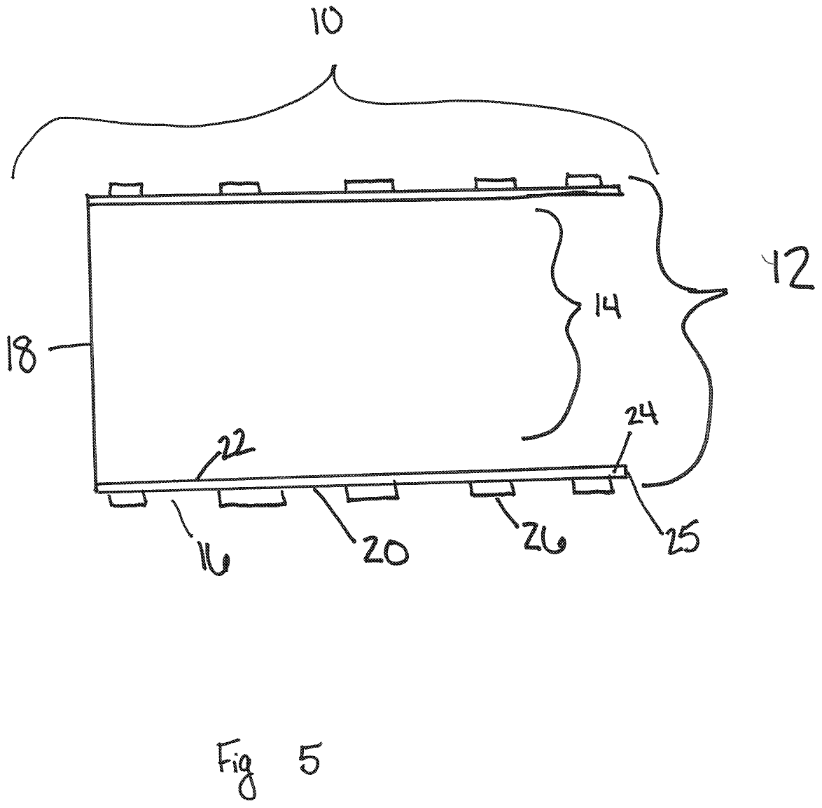

FIG. 3 shows an end view of open end 12 of endobronchial blocker 10 according to at least one embodiment of the present disclosure. Open end 12 is defined by rim 25. Shown in FIG. 3 is hollow cylinder 14 and interior surface 22. Wall thickness 24 is defined by interior surface 22 and exterior surface 20. FIG. 4 shows an end view of closed end 18 of endobronchial blocker 10 according to at least one embodiment of the present disclosure. In at least one non-limiting embodiment of the present disclosure, endobronchial blocker 10 can be inserted into a patient for treatment of a persistent airleak with open end 12 oriented toward the pleural space. In another non-limiting embodiment of the present disclosure, endobronchial blocker 10 can be inserted into a patient for treatment of a persistent airleak with closed end 18 oriented toward the pleural space.

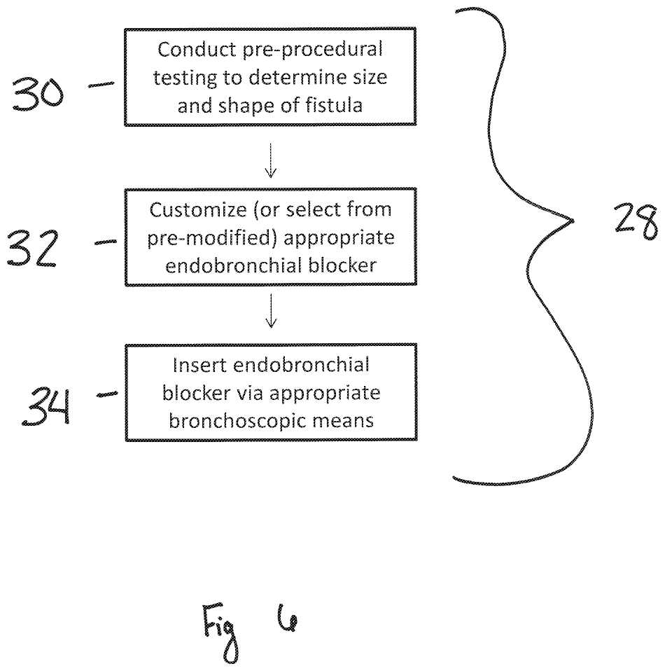

FIG. 5 shows a cross-sectional view of endobronchial blocker 10 taken along line V-V of FIG. 3. Shown in FIG. 5 is hollow tube 16 having an open end 12, closed end 18, and continuous hollow cylinder 14 between the two ends. According to the embodiment of the present disclosure shown in FIG. 5, hollow tube 16 has an interior surface 22 and an exterior surface 20 with friction enhancing elements 26 distributed on exterior surface 20. Exterior surface 20 and interior surface 22 define the perimeter wall 24, rim 25, and diameter of the hollow cylinder 14.

The size and shape of the endobronchial blocker 10 according to the present disclosure are based upon the size, shape, and location of patient's fistula, as well as the size and shape of the airway or bronchus leading to the fistula. In at least one embodiment, the size and shape of the endobronchial blocker 10 according to the present disclosure may be chosen based upon the results of pre-procedural measurements. That is, an endobronchial blocker 10 according to the present disclosure can be customized with regard to size, shape, width, wall thickness, and length, in order to substantially match the conditions of the patient's fistula, and airway or bronchus.

FIG. 6 shows a flow chart illustrating a method 28 for using an endobronchial blocker 10 to treat a persistent air leak according to at least one embodiment of the present disclosure. As shown in FIG. 6, such a method includes the step 30 of conducting pre-procedural testing to determine the airway or bronchus leading to the fistula causing the persistent air leak, as well as the size and shape and location of the fistula. This testing can be completed by any medically acceptable diagnostic method. In a particular, non-limiting embodiment, this testing can be completed using flexible or rigid bronchoscopy to inspect the airway and take appropriate measurements. As shown in FIG. 6, the method 28 also includes the step 32 of selecting the appropriate endobronchial blocker 10 to cover the fistula. This step could include specific customization, such as creating an endobronchial blocker 10 by means of 3D printing, or selecting from a variety of previously modified stents as disclosed by the present invention. As shown in FIG. 6, the method 28 also includes the step 34 of inserting endobronchial blocker 10 via appropriate bronchoscopic tools as required for the patient's individualized treatment.

While this disclosure has been described as having various embodiments, these embodiments according to the present disclosure can be further modified within the scope and spirit of this disclosure. This application is therefore intended to cover any variations, uses, or adaptations of the disclosure using its general principles. For example, any methods disclosed herein represent one possible sequence of performing the steps thereof. A practitioner may determine in a particular implementation that a plurality of steps of one or more of the disclosed methods may be combinable, or that a different sequence of steps may be employed to accomplish the same results. Each such implementation falls within the scope of the present disclosure as disclosed herein and in the appended claims. Furthermore, this application is intended to cover such departures from the present disclosure as come within known or customary practice in the art to which this disclosure pertains.

What is claimed is:

1. An endobronchial blocker for treatment of a persistent air leak, the endobronchial blocker comprising:
a perimeter wall, said perimeter wall comprising an exterior surface and an interior surface and enclosing a hollow internal cavity;
an open end, said open end comprising a rim, said rim defining an opening to said hollow internal cavity;
a closed end opposite said open end, said closed end and said perimeter wall being airtightly arranged so as to obstruct airflow into said hollow internal cavity and out of said hollow internal cavity; and
one or more friction enhancing elements on said exterior surface of said perimeter wall.

2. The endobronchial blocker of claim 1 wherein said perimeter wall comprises a flexible biocompatible material.

3. The endobronchial blocker of claim 2 wherein said flexible biocompatible material comprises an implant-grade silicone.

4. The endobronchial blocker of claim 1 wherein at least one physical characteristic of said perimeter wall is selected as a result of a patient's pre-procedural testing.

5. The endobronchial blocker of claim 4 wherein said at least one physical characteristic is selected from the group consisting of width, length, shape, and wall thickness.

6. The endobronchial blocker of claim 1 wherein said perimeter wall is cylindrical.

7. The endobronchial blocker of claim 1 wherein said perimeter wall is hour glass shaped.

8. The endobronchial blocker of claim 1 wherein said perimeter wall further comprises at least one branch.

9. The endobronchial blocker of claim 1 wherein said one or more friction enhancing elements comprise a plurality of studs distributed on said exterior surface.

10. A method of treating a persistent air leak by inserting an endobronchial blocker, comprising the steps of:
selecting a site for placement of said endobronchial blocker to treat a patient's persistent air leak comprising the steps of:
identifying an airway or a bronchus leading to said persistent air leak, and measuring at least one physical characteristic of said airway, said bronchus, or said persistent air leak;
providing an endobronchial blocker, said endobronchial blocker comprising:
a perimeter wall, said perimeter wall comprising an exterior surface and an interior surface and enclosing a hollow internal cavity,
an open end, said open end comprising a rim, said rim defining an opening to said hollow internal cavity,
a closed end opposite said open end, said closed end and said perimeter wall being airtightly arranged so as to obstruct airflow into said hollow internal cavity and out of said hollow internal cavity, and
one or more friction enhancing elements on said exterior surface of said perimeter wall;
loading said endobronchial blocker into a bronchoscope;
inserting said bronchoscope into said patient's upper airway;
maneuvering said bronchoscope to location of said bronchus or said airway selected for placement of said endobronchial blocker;
releasing said endobronchial blocker from said bronchoscope in position to occlude said persistent air leak;
monitoring clinical measurements to ensure said endobronchial blocker has reduced or eliminated said persistent air leak; and
securing said endobronchial blocker in said patient's airway.

11. The method of claim 10 wherein said endobronchial blocker comprises a flexible biocompatible material.

12. The method of claim 11 wherein said flexible biocompatible material comprises an implant-grade silicone.

13. The method of claim 10 wherein at least one physical characteristic of said endobronchial blocker is selected as a result of a patient's pre-procedural testing.

14. The method of claim 13 wherein said at least one physical characteristic is selected from the group consisting of width, length, shape, and wall thickness.

15. The method of claim 10 wherein said perimeter wall is cylindrical.

16. The method of claim 10 wherein said perimeter wall is hour glass shaped.

17. The method of claim 10 wherein said perimeter wall further comprises at least one branch.

18. The method of claim 10 wherein said one or more friction enhancing elements comprises a plurality of studs distributed on said exterior surface.

19. The method of claim 10 wherein said persistent air leak is caused by a fistula.

20. The method of claim 10 wherein said bronchoscope comprises a rigid bronchoscope.
